(19) United States
(12) Patent Application Publication
(10) Pub. No.: US 2006/0194198 A1
(43) Pub. Date: Aug. 31, 2006

(54) Use of fibrous protein fibers for chemical sensing and radiation detection

(76) Inventor: Carlo Licata, Los Angeles, CA (US)

Correspondence Address: CATALYST LAW GROUP, APC, 9710 SCRANTON ROAD, SUITE S-170, SAN DIEGO, CA, 92121, US

(21) Appl. No.: 11/174,284

(22) Filed: Jun. 29, 2005

Related U.S. Application Data
(60) Provisional application No. 60/583,728, filed on Jun. 29, 2004.

(51) Int. Cl. C12Q 1/70; C12Q 1/04; C12M 1/34

(52) U.S. Cl. 435/5; 435/34; 435/287.1; 205/777

(57) ABSTRACT

Fibrous protein fibers such as keratin fibers can be used to detect chemicals and radiation. One aspect of the invention is a method of detecting an analyte in a sample comprising the steps of: (1) providing a fiber of fibrous protein; (2) contacting the fiber of fibrous protein with an sample that may contain an analyte; (3) measuring the conductivity of the fiber of fibrous protein in the absence of contact with the sample and in the presence of contact with the sample; and (4) correlating the conductivity of the fiber of fibrous protein in the presence of contact with the sample with the conductivity of the fiber of fibrous protein in the presence of contact with a reference sample containing a known concentration of analyte to detect or determine the analyte in the sample. Another aspect of the invention is a method of detecting radiation in a sample using a fibrous protein fiber functionalized with CdTe.

### CROSS-REFERENCES

This application claims priority from Provisional Application Ser. No. 60/583,728, by Licata, entitled “Use of Fibrous Protein Fibers for Chemical Sensing and Radiation Detection”, and filed Jun. 29, 2005, which is incorporated herein in its entirety by this reference.

### BACKGROUND OF THE INVENTION

The present invention generally relates to the use of fibrous protein fibers for chemical sensing and radiation detection.

The existence of chemical and radiochemical pollution as the result of industrial processes and the use of radioisotopes has increased. Cleanup of such pollution is costly, labor-intensive, and time-consuming. Such cleanup is necessary if contaminated property, such as abandoned industrial sites, is to be returned to productive use.

The cleanup of such properties requires an easy, efficient, accurate, rapid, and cost-effective means of detecting such chemical and radiochemical pollution so that appropriate cleanup measures can be taken. This is particularly important in cases of radiochemical pollution, as even brief exposure to radiochemical pollutants can have serious long-term medical effects.

The importance of detection of chemical and radiochemical pollution has enormously increased in importance following the tragic events of Sep. 11, 2001, in New York and Washington. Many intelligence analysts now believe that Al Qaeda and other terrorist groups are interested in obtaining and using so-called “dirty bombs”. Such a “dirty bomb” contains radioactive isotopes in an explosive device that contains conventional high explosive. Detonating a dirty bomb would not cause the death and devastation that the detonation of a conventional nuclear weapon would cause, but officials and counter-terrorism experts have predicted that the detonation of such a weapon would cause some fatalities, radiation sickness, mass panic, and serious economic disruption. Concern has increased further as the result of recent terrorist attacks in Europe, such as the train bombings in Madrid, Spain. Even small amounts of radioactive contamination would send hundreds of thousands of persons to medical facilities for screening and would render large numbers of buildings uninhabitable without expensive cleanup procedures.

A number of radioactive isotopes of heavy metals could conceivably used for the production of dirty bombs. Although several isotopes could be used for the production of dirty bombs, the one that creates the most concern is ¹³⁷CS. The isotope ¹³⁷Cs is widely used, particularly in radiotherapy. In its common form, it is an ideal dirty bomb ingredient, as it is easily dispersed and binds to materials such as asphalt and concrete. The isotope ¹³⁷Cs emits beta radiation with a half-life of about 30.2 years. In recent years, there have been several seizures of large quantities of ¹³⁷Cs by law enforcement authorities as the result of illicit attempts to sell the radioisotope. Such seizures have occurred in Bangkok, Thailand, and in Tblisi, Georgia.

The International Atomic Energy Agency has estimated that 110 countries lack adequate controls over materials that could potentially be used in a dirty bomb. In the United States, the congressional General Accounting Office has estimated that there were nearly 10 million containers of radioactive waste, including medical detritus, in the United States and 49 other countries in 2003.

In addition to ¹³⁷Cs, isotopes such as ⁶⁰Co and ⁹⁰Sr also can be used as ingredients of a dirty bomb.

Accordingly, there is a need for an improved method for detection of radioactive isotopes and other pollutants, particularly heavy metals. Such a method should be accurate, easy to use, and capable of rapid detection of such radioactive isotopes and other pollutants.

### SUMMARY OF THE INVENTION

Fibrous protein fibers such as keratin fibers have semiconductive properties that make them suitable for the detection of analytes such as heavy metals and organic pollutants such as dioxin. When functionalized with CdTe, such fibers also can be used to detect radiation, such as radiation produced by radionuclides such as ¹³⁷Cs, ⁶⁰Co, or ⁹⁰Sr.

One aspect of the present invention is a method for detecting and/or determining a chemical species or analyte by using fibrous protein fibers. In general, such a method comprises the steps of:

(1) providing a fiber of fibrous protein;

(2) contacting the fiber of fibrous protein with an sample that may contain an analyte;

(3) measuring the conductivity of the fiber of fibrous protein in the absence of contact with the sample and in the presence of contact with the sample; and

(4) correlating the conductivity of the fiber of fibrous protein in the presence of contact with the sample with the conductivity of the fiber of fibrous protein in the presence of contact with a reference sample containing a known concentration of analyte to detect or determine the analyte in the sample.

Typically, the fibrous protein of the fibrous protein fiber is selected from the group consisting of keratins, collagens, fibrins, and elastins. Preferably, the fibrous protein of the fibrous protein fiber is a keratin and the keratin is selected from the group consisting of α-keratins and β-keratins. More preferably, the keratin is a β-keratin.

Typically, the β-keratin is obtained from avian feathers. Preferably, the avian feathers are obtained from a species selected from the group consisting of a chicken, a turkey, a duck, and a goose. More preferably, the avian feathers are chicken feathers.

Typically, the keratin is a naturally-occurring keratin.

The fibrous protein fiber can be incorporated or woven into a textile.

The analyte can be in gas form or in liquid form. In one alternative, the analyte is inorganic. When the analyte is inorganic, typically it is selected from the group consisting of strontium, cesium, lead, copper, cadmium, mercury, vanadium, radium, zinc, chromium, gold, silver, manganese, cobalt, nickel, and uranium. In another alternative, the analyte is a cyano or chloro complex of gold, silver, or platinum. In still another alternative, the analyte is organic. When the analyte is organic, typically it is selected from the group consisting of benzene, dioxin, polycyclic aromatic hydrocarbons, and aromatic amines.

In another alternative, the analyte can be a biological agent, such as a virus, a bacterium, or a toxin. The virus can be smallpox virus, West Nile virus, SARS virus, Ebola virus, hantavirus, or another pathogenic virus. The bacterium can be Bacillus anthracis, Yersinia pestis (the causative agent of plague), or another pathogenic bacterium. The toxin can be botulinum toxin, ricin, cholera toxin, anthrax toxin, or another biological toxin of bacterial, viral, plant, or animal origin.

Another aspect of the invention is a measuring device for detecting or determining an analyte comprising:

(1) an electrode assembly comprising a fiber of fibrous protein;

(2) means for contacting the analyte with the electrode assembly;

(3) means for measuring the conductivity of the fiber of fibrous protein in the electrode assembly, the means electrically connected to the electrode assembly and producing an output representing the conductivity of the fiber of fibrous protein, wherein the conductivity of the fiber of fibrous protein is correlated with the presence or concentration of the analyte; and

(4) means for processing the output from the means for measuring the conductivity of the fiber of fibrous protein to detect and/or determine the presence or concentration of the analyte.

The fibrous protein of the fiber is typically a keratin, as described above.

Typically, the electrode assembly is supported by a solid support. Typically, the solid support is rigid plastic. Preferably, the rigid plastic is tetrafluoropolyethylene.

Typically, the electrode assembly further includes platinum electrodes at each end of the fiber of fibrous protein.

The data processor can be a dedicated data processing module controlled by firmware. Alternatively, the data processor can be a computer controlled by software.

Another aspect of the invention is measuring device for detecting and/or determining an analyte comprising:

(1) an electrode assembly comprising:
(a) a rigid support;
(b) a fiber of fibrous protein supported by the rigid support;
(c) an inlet for a sample so that the sample can contact the fiber of fibrous protein;
(d) an outlet for the sample; and
(e) two conductive electrodes attached to each end of the fiber of fibrous protein;

(2) a conductivity measuring unit electrically connected to the two conductive electrodes, the conductivity measuring unit producing an output representing the conductivity of the fiber of fibrous protein, wherein the conductivity of the fiber of fibrous protein is correlated with the presence or concentration of the analyte; and

(3) a data processor to which the output of the conductivity measuring unit is fed, the data processor producing a detectable indication of the presence or quantity of the analyte.

In this device, the analyte can be in gas form and the inlet for the sample then supplies the analyte in gas form to the electrode assembly. Alternatively, the analyte can be in liquid form and the inlet for the sample then supplies the analyte in liquid form to the electrode assembly.

Another aspect of the present invention is a fibrous protein fiber functionalized with CdTe to a sufficient extent that the fibrous protein fiber emits a detectable signal as a free electron plus a hole when the functionalized fibrous protein fiber is exposed to ionizing radiation. The fibrous protein of the fibrous protein fiber is typically a keratin, as described above. The fibrous protein of the fibrous protein fiber can be incorporated or woven into a textile.

Yet another aspect of the present invention is a method of functionalizing a fibrous protein fiber electrochemically with CdTe comprising the step of exposing a fibrous protein fiber to a substantially constant voltage in an aqueous solution containing CdSO₄₋ and HTeO₂⁺ to deposit CdTe on the fiber.

Yet another aspect of the present invention is a device for functionalizing a fibrous protein fiber electrochemically with CdTe comprising:

(1) a container for a CdSO₄₋ and HTeO₂⁺ -containing aqueous solution;

(2) an electrode including a fibrous protein fiber placed in the container;

(3) a reference electrode placed in the container;

(4) a platinum counter-electrode placed in the container; and

(5) a potentiostat electrically connected to the electrode including the fibrous protein fiber, the reference electrode, and the platinum counter-electrode, the potentiostat maintaining a substantially constant voltage.

Yet another aspect of the present invention is a method of detecting and/or determining ionizing radiation comprising the steps of:

(1) exposing a fibrous protein fiber functionalized with CdTe to a sufficient extent that the fibrous protein fiber emits a detectable signal as a free electron plus a hole when the functionalized fibrous protein fiber is exposed to ionizing radiation to a dose of ionizing radiation; and

(2) detecting the signal emitted from the functionalized protein fiber to detect and/or determine the ionizing radiation.

In this method, typically, the radiation is emitted by a radionuclide. The radionuclide is typically one of ¹³⁷Cs, ⁶⁰CO, or ⁹⁰Sr. In one particularly useful alternative, the radionuclide is ¹³⁷Cs.

Yet another aspect of the present invention is a device for detecting and/or determining ionizing radiation comprising:
(a) a fibrous protein fiber functionalized with CdTe to a sufficient extent that the fibrous protein fiber emits a detectable signal as a free electron plus a hole when the functionalized fibrous protein fiber is exposed to ionizing radiation; and
(b) processing means to process the detectable signal when the functionalized fibrous protein fiber is exposed to ionizing radiation so that ionizing radiation is detected and/or determined.

Still another aspect of the present invention is a device for detecting and/or determining ionizing radiation comprising:

(1) a first fibrous protein fiber functionalized with CdTe to a sufficient extent that the fibrous protein fiber emits a detectable signal as a free electron plus a hole when the functionalized fibrous protein fiber is exposed to ionizing radiation;

(2) a second fibrous protein fiber functionalized with CdTe to a sufficient extent that the fibrous protein fiber emits a detectable signal as a free electron plus a hole when the functionalized fibrous protein fiber is exposed to ionizing radiation;

(3) a first and second preamplifier each driven by output from the first fibrous protein fiber functionalized with CdTe;

(4) a third and fourth preamplifier each driven by output from the second fibrous protein fiber functionalized with CdTe;

(5) a first differential amplifier driven by output from the first and second preamplifiers;

(6) a second differential amplifier driven by output from the third and fourth preamplifiers;

(7) a first shaping amplifier driven by output from the first differential amplifier;

(8) a second shaping amplifier driven by output from the second differential amplifier;

(9) a first gate delay driven by output from the first shaping amplifier;

(10) a second gate delay driven by output from the second shaping amplifier;

(11) a first linear gate driven by output from the first shaping amplifier and from the second gate delay;

(12) a second linear gate driven by output from the second shaping amplifier and from the first gate delay;

(13) a sum amplifier driven by output from the first and second linear gates; and

(14) a data processing device fed by output from the sum amplifier; wherein the device functions as a coincidence counter.

### BRIEF DESCRIPTION OF THE DRAWINGS

The following invention will become better understood with reference to the specification, appended claims, and accompanying drawings, where:

FIG. 1 is a scanning electron microscope (SEM) picture of keratin protein fibers.

FIG. 2 is a SEM picture of keratin protein fiber showing the porous network.

FIG. 3 is a high magnification SEM image of keratin protein fiber showing the nanosized pores.

FIG. 4 is an atomic force microscope (AFM) picture of keratin protein fibers showing the microstructural morphology.

FIG. 5 is a SEM image of keratin quill showing the organized structure.

FIG. 6 is a SEM image of keratin quill revealing the spacing of micropores.

FIG. 7 is a graph showing the zeta potential of finely ground keratin protein fiber in the presence of released calcium.

FIG. 8 is a diagram showing the structure of keratin protein fiber showing the multiple functionalities: (a) electrostatic interaction; (b) hydrogen bonding; (c) hydrophobic interactions; and (d) disulfide linkages.

FIG. 9 is a schematic illustration of the working principle of a semiconductor-type radiation detector using functionalized fibrous protein fibers.

FIG. 10 is a schematic setup for the functionalization of fibrous protein fibers by an electrochemical process.

FIG. 11 is a schematic setup for the detection of chemicals using fibrous protein fibers by monitoring the change in conductivity of the fibers by absorption.

FIG. 12 is a block diagram of electronics proposed for two-detector functionalized fibrous protein fiber radiation measurements.

### DETAILED DESCRIPTION OF THE INVENTION

The following detailed description is of the best currently contemplated modes of carrying out the invention. The description is not to be taken in a limiting sense, but is made merely for the purpose of illustrating the general principles of the invention, since the scope of the invention is best defined by the appended claims.

One aspect of the present invention is a method for detecting and/or determining a chemical species or analyte by using fibrous protein fibers. The chemical species or analyte to be detected can be in gas or liquid form. The chemical species can be inorganic or organic. For example, the chemical species can be a heavy metal such as strontium, cesium, lead, copper, cadmium, mercury, vanadium, radium, zinc, chromium, gold, silver, manganese, cobalt, nickel, or uranium. In another alternative, the chemical species can be a cyano or chloro complex of gold, silver, or platinum. Alternatively, the chemical species can be an organic species such as an aliphatic or aromatic compound. Organic species that can be detected by methods according to the present invention include benzene, dioxin, polycyclic aromatic hydrocarbons, and aromatic amines. These compounds are suspected carcinogens.

In still another alternative, the analyte can be a biological agent, such as a virus, a bacterium, or a toxin. The virus can be smallpox virus, West Nile virus, SARS virus, Ebola virus, hantavirus, or another pathogenic virus. The bacterium can be Bacillus anthracis, Yersinia pestis (the causative agent of plague), or another pathogenic bacterium, such as the causative agents of tuberculosis, syphilis, or other bacterial diseases. The toxin can be botulinum toxin, ricin, cholera toxin, anthrax toxin, or another biological toxin of bacterial, viral, plant, or animal origin.

As used herein, the terms “detected” or “detecting” refer to a qualitative indication of a substance or energy to be detected, such as a pollutant, a biological agent, or ionizing radiation, while the terms “determining” or “determined” refer to a quantitative or semiquantitative indication of a substance or energy to be detected.

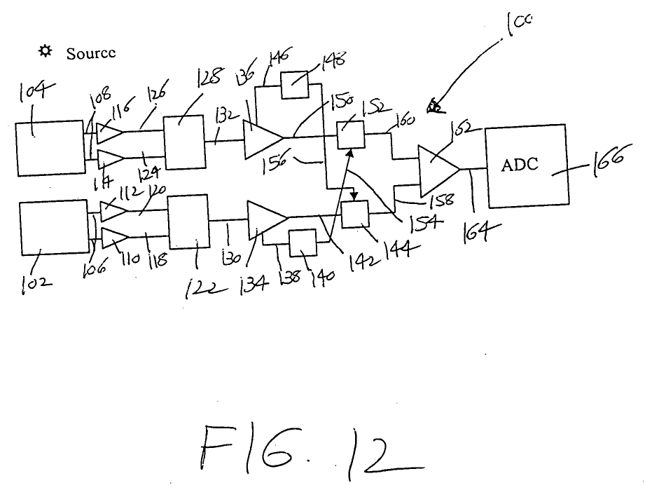

The conductivity of fibrous protein fibers changes with adsorption of these chemical species. The change in conductivity can be measured by conventional electrical measurements, as is shown in FIG. 11.

In general, such a method comprises the steps of:

(1) providing a fiber of fibrous protein;

(2) contacting the fiber of fibrous protein with an sample that may contain an analyte;

(3) measuring the conductivity of the fiber of fibrous protein in the absence of contact with the sample and in the presence of contact with the sample; and

(4) correlating the conductivity of the fiber of fibrous protein in the presence of contact with the sample with the conductivity of the fiber of fibrous protein in the presence of contact with a reference sample containing a known concentration of analyte to detect or determine the analyte in the sample.

A suitable setup for the performance of this measurement is shown in FIG. 11. The measuring device 10 comprises an electrode assembly 12, a conductivity measuring unit 14 and a data processor 16. The electrode assembly 12 includes a rigid support 18 that supports a fiber 20 of fibrous protein and two conductive electrodes 22 and 24. The rigid support 18 is typically constructed of polytetrafluoroethylene (PTFE) or another rigid plastic. The conductive electrodes 22 and 24 are typically constructed of platinum, but can be constructed of any highly conductive metal that does not react with any substance present in the sample. The electrode assembly further includes an inlet 26 for the sample so that the sample can contact the fiber 20 and an outlet 28 for the sample, allowing the sample to flow through the electrode assembly 12. Leads 30 and 32 connect the electrodes 22 and 24 with the conductivity measuring unit 14. The output from the conductivity measuring unit is fed to the data processor 16. The data processor 16 can be a computer controlled by software or a dedicated data processing module controlled by firmware. The output of the data processor 16 can be displayed on a screen, printed, or stored in digital form in a conventional storage device such as a hard drive, floppy disk, or CD-ROM.

Accordingly, another aspect of the present invention is a measuring device for detecting or determining an analyte. In general, a measuring device for detecting or determining an analyte according to this aspect of the present invention comprises:

(1) an electrode assembly comprising a fiber of fibrous protein;

(2) means for contacting the analyte with the electrode assembly;

(3) means for measuring the conductivity of the fiber of fibrous protein in the electrode assembly, the means electrically connected to the electrode assembly and producing an output representing the conductivity of the fiber of fibrous protein, wherein the conductivity of the fiber of fibrous protein is correlated with the presence or concentration of the analyte; and

(4) means for processing the output from the means for measuring the conductivity of the fiber of fibrous protein to detect and/or determine the analyte.

Typically, as described above, the electrode assembly is supported by a rigid support and includes platinum electrodes at each end of the fiber of fibrous protein. The rigid support can be a rigid plastic such as polytetrafluoroethylene (PTFE). The means for measuring the conductivity of the fiber of fibrous protein is typically connected with the electrode assembly by leads, as is conventionally known in the art. The means for measuring the conductivity of the fiber of fibrous protein can be a digital multimeter or another conductivity meter; such devices are again well known in the art and need not be described further in detail. The data processor can be a computer controlled by software or a dedicated data processing module controlled by firmware, as described above. The output of the data processor can be displayed on a screen, printed, or stored in digital form in a conventional storage device such as a hard drive, floppy disk, or CD-ROM. Again, such data processor and storage devices are well known in the art and need not be described further here.

Typically, the fibrous protein of the fibrous protein fiber is a keratin, a collagen, a fibrin, or an elastin.

In one alternative, the fibrous protein is a keratin.

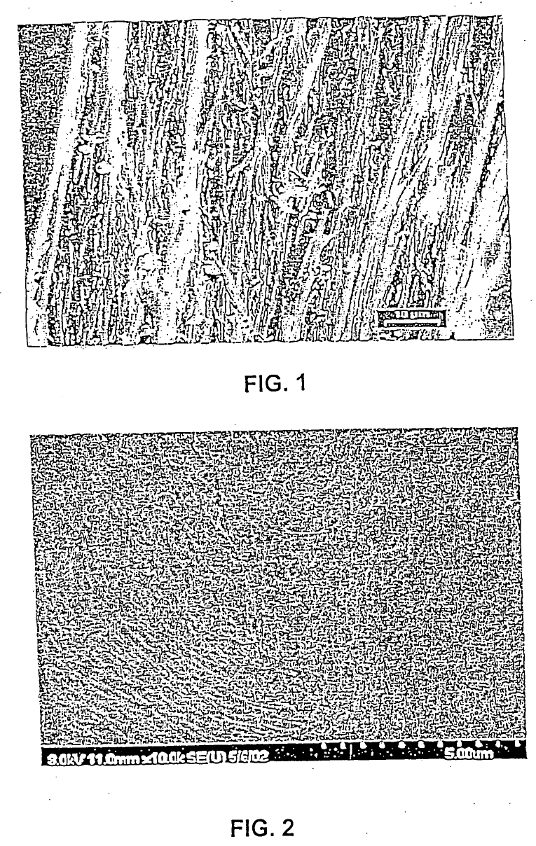

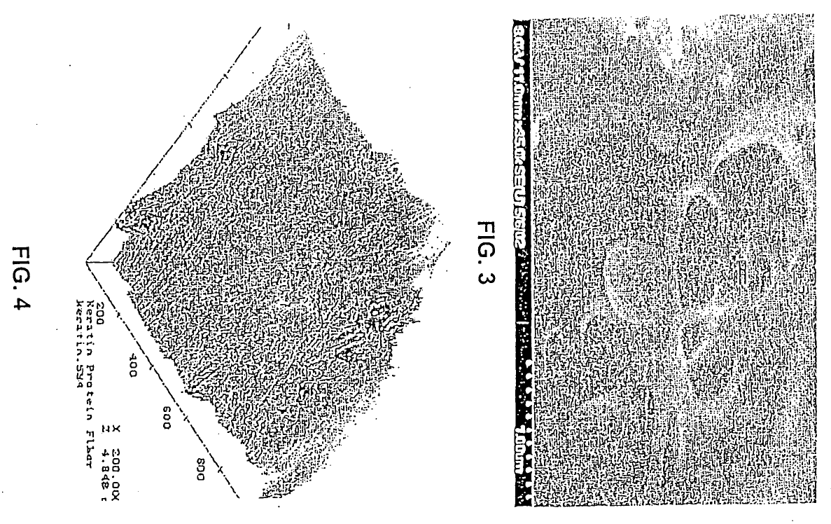

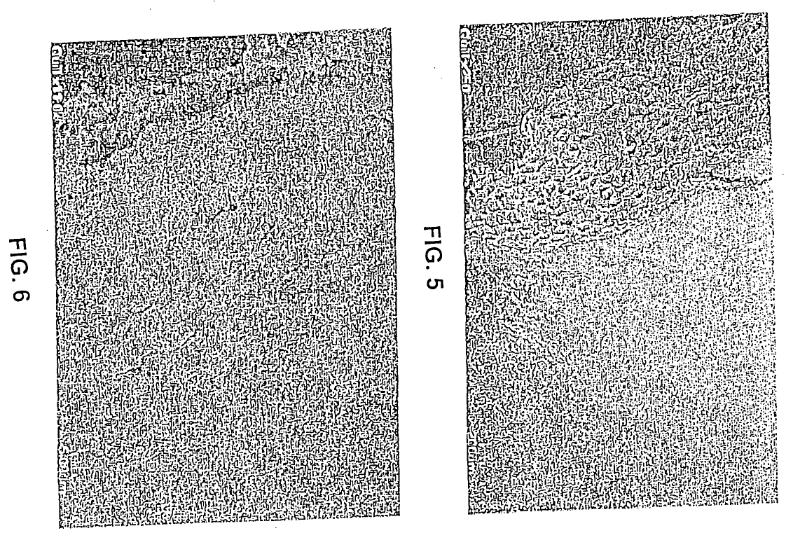

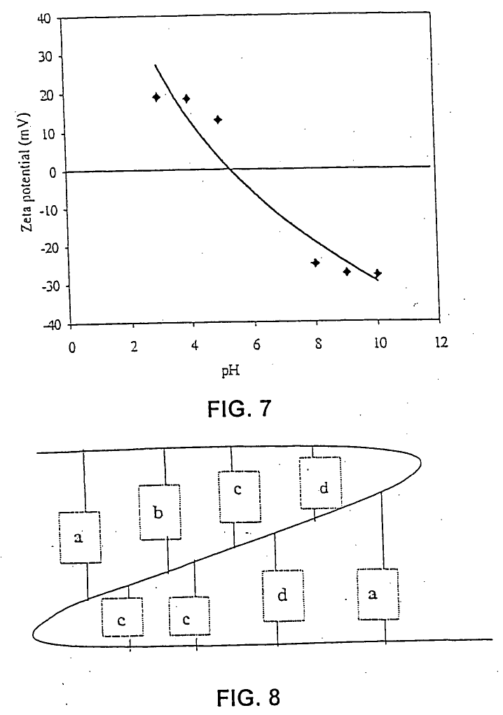

Keratin protein fibers have an intricate network of connective fibrous structure. A scanning electron microscope (SEM) picture of keratin protein fibers is shown in FIG. 1. The length of a single keratin fiber is approximately 200 microns and the maximum diameter of the fiber is 25 to 50 microns. The fiber fraction of the feather material has an organized microstructure and a nano-porous network with pores in the size range of 0.05 to 0.1 microns as shown in FIGS. 2 and 3. An atomic force microscope (AFM) picture of keratin protein fibers is shown in FIG. 4 and shows that the dimension of the fibers is in the nanoscale range. The high resolution AFM picture shows that each fiber is composed of numerous fine fibrous strands. Quill is hard and has an organized structure as shown in FIG. 5. However, the frequency of micropores (except in the detached cross-section) found in the fiber is indicated by FIG. 6. The surface area of keratin protein fiber, as determined by BET, is around 11 m²/g. Fourier Transform Infrared Spectroscopic (FTIR) analysis confirmed the presence of C—H (3076.065 cm-⁻¹), COOH (1653.487 and 1637.178 cm⁻¹), N—H (1540.583 cm⁻¹), C—S (1075.980 and 1073.819 cm⁻¹) and S═S (617.715 cm⁻¹) groups in the keratin fibers. The zeta potential of the keratin fiber as a function of pH is shown in FIG. 7. At a pH above 5, the keratin protein is negatively charged, and at acidic pH it is positively charged. This behavior of keratin protein is due to the presence of various acidic and basic functional groups.

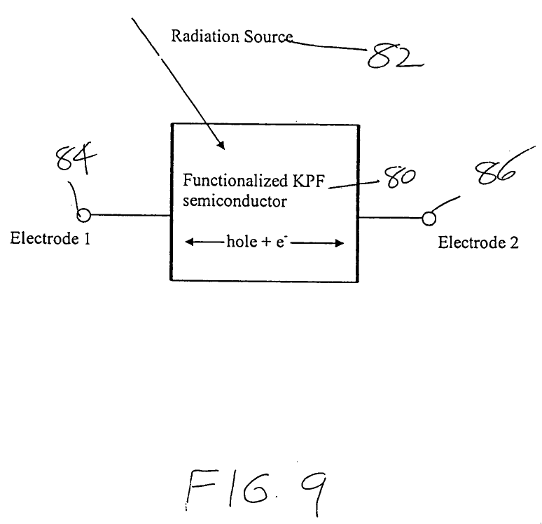

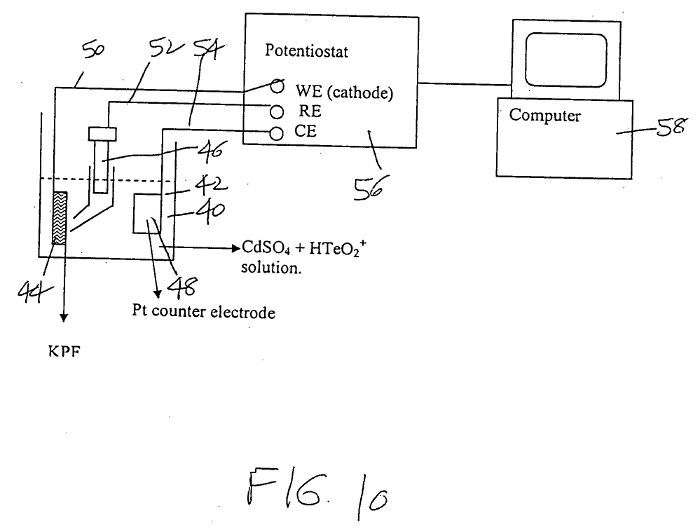

Amino acid analysis of the keratin fiber revealed a characteristic abundance of cysteine residues (7-20% of the total amino acid residues). These cysteine residues are oxidized to give inter- and intra-molecular disulfide bonds, which create the mechanically strong three-dimensionally linked network of keratin fiber. Each polypeptide chain in the feather keratin has a central helical section with a less regular region at each end. It has cross-linking hydrogen bonds formed between two parts of the protein chain that can be far apart. FIG. 8 illustrates schematically some of the functional groups present in the helical structure of the keratin protein and their resulting interactions: (a) electrostatic interactions; (b) hydrogen bonding; (c) hydrophobic interactions; and (d) disulfide linkages.

The combination of nano-fibrous structure and functional groups makes protein fibers an excellent biosorption material. Such protein fibers have the desirable properties recited above. These properties can be modified according to the desired use of the composition incorporating the protein fibers.

The structure and properties of keratins are described in D. Voet & J.G. Voet, “Biochemistry” (2d ed., John Wiley & Sons, New York, 1995), pp. 153-155, incorporated herein by this reference. Keratins are divided into two classes, α-keratins and β-keratins. The α-keratins are found in mammals, and the β-keratins are found in birds and reptiles.

Fibrous protein fibers used in devices and methods according to the present invention can comprise keratins obtained from avian feathers, including poultry such as chicken, turkey, duck, and goose, and other commercially available poultry species, as well as feathers from other birds such as pigeons. Alternatively, fibrous protein fibers used in devices and methods according to the present invention can comprise keratins obtained from other sources such as wool, egg shell membrane, silk, spider web, animal hair, human hair, animal nail, human nail, animal skin, and human skin, or their components. As used herein, the term “keratin” includes both naturally-occurring keratin and keratin that has been modified by reactions such as acetylation, phosphorylation, hydroxylation, glycosylation, and other chemical reactions that can occur on the functional groups of the keratin proteins. As used herein, the term “keratin” further includes muteins of keratin that are produced by genetic engineering techniques well-known in the art, such as site-specific mutagenesis, and fusion proteins that incorporate keratins, as long as such fusion proteins remain fibrous. However, it is generally preferable to use naturally-occuring unmodified keratins in devices and methods according to the present invention.

Alternatively, fibrous protein fibers used in devices and methods according to the present invention can comprise other fibrous proteins, either naturally-occurring fibrous proteins or chemically-modified or genetically-engineered fibrous proteins. Naturally-occurring fibrous proteins include, but are not limited to, fibroin, collagen, and elastin. Fibrous proteins are described in D. Voet & J.G. Voet, “Biochemistry” (2d ed., John Wiley & Sons, New York, 1995), pp. 153-162, incorporated herein by this reference.

In one preferred embodiment of this alternative, the fibrous protein is a collagen or an elastin. In this preferred embodiment, the fibrous protein of the fibrous protein fiber is typically obtained from a domestic mammal, such as a cow, a pig, a sheep, or a goat, or from the non-feather portion of a bird, such as a chicken, a turkey, a duck, or a goose. The use of these fibrous proteins provides an economically feasible alternative route for the disposal of animal waste products. As used herein, the terms “collagen” or “elastin” further includes muteins of collagen or elastin that are produced by genetic engineering techniques well-known in the art, such as site-specific mutagenesis, and fusion proteins that incorporate collagen or elastin, as long as such fusion proteins remain fibrous. However, it is generally preferable to use naturally-occurring unmodified collagens or elastins in compositions according to the present invention.

Preferably, if the fibrous protein of the fibrous protein fiber is a keratin, the keratin protein is from a feather, such as that of a chicken. More preferably, the keratin protein is from the fiber portion of the feather as compared to the quill portion. Thus, the keratin protein may be essentially only the fiber portion of a chicken feather that has been separated from the quill portion. What is meant by “essentially” is that the keratin protein is characterized by a fiber to quill weight ratio of at least about 1:1. The manner of preparing and then separating the fiber from the quill does not comprise a part of the present invention and may be accomplished by known methods such as that described in U.S. Pat. No. 5,705,030, incorporated herein by this reference.

The size of the fibrous protein particles, such as keratin protein particles, collagen protein particles, or elastin protein particles, provided may vary from about 2 mm to 0.01 mm. Preferably, however, the size is between about 2 mm to 0.1 mm, and more preferably between about 1 mm to 0.1 mm.

The fibrous protein fiber can be incorporated or woven into a textile.

Another embodiment of the present invention is devices and methods for detecting and/or determining the presence or concentration of radiation or radioisotopes. The semiconducting properties of fibrous protein fibers, particularly keratin protein fibers, are comparable to those of silicon. The disadvantage of using silicon in radiation detectors is that it is poor in detecting energies greater than about 40 keV. By functionalizing fibrous protein fibers, particularly keratin protein fibers, large band gap semiconducting materials for detecting higher energy radiation can be obtained.

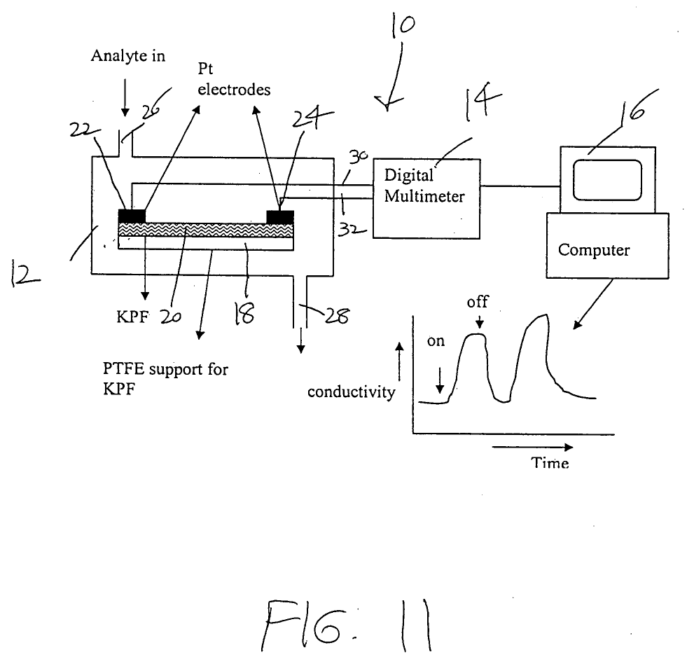

Typically, fibrous protein fibers, particularly keratin protein fibers, are functionalized with CdTe electrochemically using a CdSO₄- and HTeO₂⁺-containing aqueous solution. An apparatus for doing so is shown in FIG. 10. A container 40 holds a CdSO₄- and HTeO₂⁺-containing aqueous solution 42. In the container 40 are placed an electrode 44 of a fibrous protein fiber, such as a keratin protein fiber, a reference electrode 46, and a platinum counter electrode 48. Conductors 50, 52, and 54 are placed between the electrode 44 of the fibrous protein fiber, the reference electrode 46, and the platinum counter electrode 48 and a potentiostat 56. The potentiostat 56 is controlled by a computer 58 or other data processing device to maintain a constant potential.

Accordingly, another aspect of the invention is a method of functionalizing a fibrous protein fiber electrochemically with CdTe comprising the step of exposing a fibrous protein fiber to a substantially constant voltage in an aqueous solution containing CdSO₄- and HTeO₂⁺to deposit CdTe on the fiber.

Another aspect of the invention is a device for functionalizing a fibrous protein fiber electrochemically with CdTe comprising:

(1) a container for a CdSO₄- and HTeO₂⁺-containing aqueous solution;

(2) an electrode including a fibrous protein fiber placed in the container;

(3) a reference electrode placed in the container;

(4) a platinum counter-electrode placed in the container; and

(5) a potentiostat electrically connected to the electrode including the fibrous protein fiber, the reference electrode, and the platinum counter-electrode, the potentiostat maintaining a substantially constant voltage.

A schematic illustration of the working principle of the fibrous protein fiber radiation detector is shown in FIG. 9. In FIG. 9, a functionalized fibrous protein fiber semiconductor 80 is subjected to a radiation source 82. The functionalized fibrous protein fiber semiconductor 80 is connected to a first electrode 84 and a second electrode 86. The radiation source generates a hole plus e⁻, which are detected by the first and second electrodes 84 and 86.

A block diagram of electronics for two-detector functionalized fibrous protein fiber radiation measurements is shown in FIG. 12. The device 100 includes a first functionalized fibrous protein fiber 102 and a second functionalized fibrous protein fiber 104. The first and second functionalized fibrous protein fibers 102 and 104 are connected by leads 106 and 108 to two sets of preamplifiers, a first set 110 and 112 for the first functionalized protein fiber 102 and a second set 114 and 116 for the second functionalized protein fiber 104. The first set of preamplifiers 110 and 112 is connected by leads 118 and 120 to a first differential amplifier 122. The second set of preamplifiers 114 and 116 is connected by leads 124 and 126 to a second differential amplifier 128. The first and second differential amplifiers are then connected by leads 130 and 132 to first and second shaping amplifiers 134 and 136. The output from the first shaping amplifier 134 is then fed through a lead 138 to a first gate delay 140 and through a lead 142 to a first linear gate 144. The output from the second shaping amplifier is similarly then fed through a lead 146 to a second gate delay 148 and through a lead 150 to a second linear gate 152. The output from the first gate delay 140 is then fed through a lead 154 to the second linear gate 152. Similarly, the output from the second gate delay 148 is then fed through a lead 156 to the first linear gate 144. The outputs from the first and second linear gates 144 and 152 are then fed through leads 158 and 160 to a sum amplifier 162. Output from the sum amplifier 162 is then fed through lead 164 to a data analysis device 166, which can be a computer driven by software or a dedicated data processor driven by firmware. These amplification and signal processing devices are conventional in the art and need not be described further here in detail. The device described herein is known in the art as a coincidence counter. Such a device provides a positive signal only if both the first and second functionalized protein fibers receive a signal simultaneously. The purpose of using coincidence counting is to reduce the background count and to improve the signal-to-noise ratio (S/N).

Devices according to this embodiment of the present invention can detect radiation produced by any naturally-occurring or artificially-produced radionuclide, but are particularly suited to the detection of radiation produced by one of ¹³⁷Cs, ⁶⁰Co, and ⁹⁰Sr. Devices according to this embodiment of the present invention are most particularly suited to the detection of radiation produced by ¹³⁷Cs.

Accordingly, one aspect of this embodiment of the present invention is a fibrous protein fiber functionalized with CdTe to a sufficient extent that the fibrous protein fiber emits a detectable signal as a free electron plus a hole when the functionalized fibrous protein fiber is exposed to ionizing radiation. As detailed above, preferably the fibrous protein fiber functionalized with CdTe is a keratin fiber, but other fibrous proteins can be incorporated into the fiber. The fibrous protein fiber can be woven or incorporated into a textile.

The radionuclide that produces the ionizing radiation is typically one of ¹³⁷Cs, ⁶⁰Co, and ⁹⁰Sr. However, preferably, the radionuclide that produces the ionizing radiation is ¹³⁷Cs.

Another aspect of this embodiment of the present invention is a method of detecting and/or determining ionizing radiation comprising the steps of:

(1) exposing a fibrous protein fiber functionalized with CdTe to a sufficient extent that the fibrous protein fiber emits a detectable signal as a free electron plus a hole when the functionalized fibrous protein fiber is exposed to ionizing radiation to a dose of ionizing radiation; and

(2) detecting the signal emitted from the functionalized protein fiber to detect and/or determine the ionizing radiation.

Yet another aspect of the present invention is a device for detecting and/or determining ionizing radiation comprising:

(1) a fibrous protein fiber functionalized with CdTe to a sufficient extent that the fibrous protein fiber emits a detectable signal as a free electron plus a hole when the functionalized fibrous protein fiber is exposed to ionizing radiation; and

(2) processing means to process the detectable signal when the functionalized fibrous protein fiber is exposed to ionizing radiation so that ionizing radiation is detected and/or determined.

Still another aspect of the present invention is a device for detecting and/or determining ionizing radiation comprising:

(1) a first fibrous protein fiber functionalized with CdTe to a sufficient extent that the fibrous protein fiber emits a detectable signal as a free electron plus a hole when the functionalized fibrous protein fiber is exposed to ionizing radiation;

(2) a second fibrous protein fiber functionalized with CdTe to a sufficient extent that the fibrous protein fiber emits a detectable signal as a free electron plus a hole when the functionalized fibrous protein fiber is exposed to ionizing radiation;

(3) a first and second preamplifier each driven by output from the first fibrous protein fiber functionalized with CdTe;

(4) a third and fourth preamplifier each driven by output from the second fibrous protein fiber functionalized with CdTe;

(5) a first differential amplifier driven by output from the first and second preamplifiers;

(6) a second differential amplifier driven by output from the third and fourth preamplifiers;

(7) a first shaping amplifier driven by output from the first differential amplifier;

(8) a second shaping amplifier driven by output from the second differential amplifier;

(9) a first gate delay driven by output from the first shaping amplifier;

(10) a second gate delay driven by output from the second shaping amplifier;

(11) a first linear gate driven by output from the first shaping amplifier and from the second gate delay;

(12) a second linear gate driven by output from the second shaping amplifier and from the first gate delay;

(13) a sum amplifier driven by output from the first and second linear gates; and

(14) a data processing device fed by output from the sum amplifier.

This device is a coincidence counter. Such a device provides a positive signal only if both the first and second functionalized protein fibers receive a signal simultaneously. The purpose of using coincidence counting is to reduce the background count and to improve the signal-to-noise ratio (S/N).

The electronic components used in this device are well-known in the art and need not be described further in detail. The data processing device fed by output from the sum amplifier can be a computer controlled by software or a dedicated data processing module controlled by firmware, as described above. The output of the data processor can be displayed on a screen, printed, or stored in digital form in a conventional storage device such as a hard drive, floppy disk, or CD-ROM. Again, such data processing and storage devices are well known in the art and need not be described further here.

The radionuclide that produces the ionizing radiation for detection by this device is typically one of ¹³⁷Cs, ⁶⁰Co, and ⁹⁰Sr. However, preferably, the radionuclide that produces the ionizing radiation is ¹³⁷Cs.

### ADVANTAGES OF THE INVENTION

This invention provides a new and efficient way of detecting organic and inorganic pollutants, biological agents, and ionizing radiation, particularly ionizing radiation produced by radionuclides such as ¹³⁷Cs, ⁶⁰Co, and ⁹⁰Sr. These detection methods are particularly relevant in detecting radiation that might be associated with a device such as a “dirty bomb”.

The inventions illustratively described herein can suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference. I claim:

1. A method of detecting an analyte in a sample comprising the steps of:
(a) providing a fiber of fibrous protein;
(b) contacting the fiber of fibrous protein with an sample that may contain an analyte;
(c) measuring the conductivity of the fiber of fibrous protein in the absence of contact with the sample and in the presence of contact with the sample; and
(d) correlating the conductivity of the fiber of fibrous protein in the presence of contact with the sample with the conductivity of the fiber of fibrous protein in the presence of contact with a reference sample containing a known concentration of analyte to detect or determine the analyte in the sample.

2. The method of claim 1 wherein the fibrous protein of the fibrous protein fiber is selected from the group consisting of keratins, collagens, fibrins, and elastins.

3. The method of claim 2 wherein the fibrous protein is a keratin and the keratin is selected from the group consisting of α-keratins and β-keratins.

4. The method of claim 3 wherein the keratin is obtained from chicken feathers.

5. The method of claim 1 wherein the analyte is inorganic.

6. The method of claim 5 wherein the analyte is selected from the group consisting of strontium, cesium, lead, copper, cadmium, mercury, vanadium, radium, zinc, chromium, gold, silver, manganese, cobalt, nickel, and uranium, and a cyano or chloro complex of gold, silver, or platinum.

7. The method of claim 1 wherein the analyte is organic.

8. The method of claim 7 wherein the analyte is selected from the group consisting of benzene, dioxin, polycyclic aromatic hydrocarbons, and aromatic amines.

9. The method of claim 1 wherein the analyte is a biological agent.

10. The method of claim 9 wherein the biological agent is selected from the group consisting of a virus, a bacterium, and a toxin.

11. A measuring device for detecting or determining an analyte comprising:
(a) an electrode assembly comprising a fiber of fibrous protein;
(b) means for contacting the analyte with the electrode assembly;
(c) means for measuring the conductivity of the fiber of fibrous protein in the electrode assembly, the means electrically connected to the electrode assembly and producing an output representing the conductivity of the fiber of fibrous protein, wherein the conductivity of the fiber of fibrous protein is correlated with the presence or concentration of the analyte; and
(d) means for processing the output from the means for measuring the conductivity of the fiber of fibrous protein to detect and/or determine the presence or concentration of the analyte.

12. The measuring device of claim 11 wherein the fibrous protein of the fibrous protein fiber is selected from the group consisting of keratins, collagens, fibrins, and elastins.

13. The measuring device of claim 12 wherein the fibrous protein is a keratin and the keratin is selected from the group consisting of α-keratins and β-keratins.

14. The measuring device of claim 13 wherein the keratin is obtained from chicken feathers.

15. A measuring device for detecting and/or determining an analyte comprising:
(a) an electrode assembly comprising:
(i) a rigid support;
(ii) a fiber of fibrous protein supported by the rigid support;
(iii) an inlet for a sample so that the sample can contact the fiber of fibrous protein;
(iv) an outlet for the sample; and
(v) two conductive electrodes attached to each end of the fiber of fibrous protein;
(b) a conductivity measuring unit electrically connected to the two conductive electrodes, the conductivity measuring unit producing an output representing the conductivity of the fiber of fibrous protein, wherein the conductivity of the fiber of fibrous protein is correlated with the presence or concentration of the analyte; and
(c) a data processor to which the output of the conductivity measuring unit is fed, the data processor producing a detectable indication of the presence or quantity of the analyte.

16. The measuring device of claim 15 wherein the fibrous protein of the fibrous protein fiber is selected from the group consisting of keratins, collagens, fibrins, and elastins.

17. The measuring device of claim 16 wherein the fibrous protein is a keratin and the keratin is selected from the group consisting of β-keratins and β-keratins.

18. The measuring device of claim 17 wherein the keratin is obtained from chicken feathers.

19. A fibrous protein fiber functionalized with CdTe to a sufficient extent that the fibrous protein fiber emits a detectable signal as a free electron plus a hole when the functionalized fibrous protein fiber is exposed to ionizing radiation.

20. The fibrous protein fiber of claim 19 wherein the fibrous protein of the fibrous protein fiber is selected from the group consisting of keratins, collagens, fibrins, and elastins.
